(19) United States
(12) Patent Application Publication
(10) Pub. No.: US 2026/0203420 A1
(43) Pub. Date: Jul. 16, 2026

(54) INFERENCE OF SECURE IMAGING DATA

(71) Applicant: CANON MEDICAL SYSTEMS CORPORATION, Otawara-shi (JP)

(72) Inventors: Catherine TAYLOR, Edinburgh (GB); Paul THOMSON, Edinburgh (GB)

(73) Assignee: CANON MEDICAL SYSTEMS CORPORATION, Otawara-shi (JP)

(21) Appl. No.: 19/016,570

(22) Filed: Jan. 10, 2025

(51) Int. Cl. G06F 21/60; G16H 30/40; G16H 50/20; H04L 9/00; H04L 9/30

(52) U.S. Cl. CPC G06F 21/602; G16H 30/40; G16H 50/20; H04L 9/008; H04L 9/3073

(57) ABSTRACT

A medical information processing apparatus comprising a processing circuitry configured to receive imaging data; apply a first part of an inference model to the imaging data to obtain intermediate analysis results; encrypt the intermediate analysis results based on a homomorphic encryption scheme and send the encrypted intermediate analysis results to a remote server for processing with a second part of an inference model to result in encrypted analysis results. The processing circuitry is further configured to receive the encrypted analysis results from the remote server; and decrypt the encrypted analysis results to obtain analysis results.

### FIELD

The present invention relates to the inference of imaging data, and in particular to the inference of secure medical imaging data on remote servers.

### BACKGROUND

Imaging data can be processed by models that perform inference tasks such as image classification or image segmentation. Image classification is the process of categorizing an image based on its content. Image segmentation is the process of partitioning an image into multiple image segments based on characteristics of the respective image segments such as colour, intensity, and texture. These models may be artificial intelligence methods with architectures comprising one or more of neural networks, transformers, encoders and decoders.

An encoder-decoder framework is a commonly used architecture in deep learning. The encoder takes in input data (e.g., a medical image) and creates a lower dimensional and efficient latent representation by extracting useful features. The latent representation has a fixed size, while the size of the input data can vary. The decoder then takes the new representation and generates the required output data (e.g., a segmentation mask). Again, the output can have varied size.

Image analysis models can be offered by third party companies as a service. These models may be proprietary custom solutions and a third party may public restrict access to these models by storing them on one or more private servers.

Image analysis models are routinely used in a medical context to diagnose and detect pathologies. These models can be applied to medical imaging data which is generated for a patient as part of a clinical analysis procedure. The protection of patient medical data to maintain patient privacy is a key concern for healthcare providers. As a result, health care providers are often unwilling for patient medical data to leave their secure servers.

The need to protect, on one hand, the privacy of patient medical data, and on the other hand, the intellectual property of third party models, can make it challenging for patient medical data to be securely analysed by third party proprietary models.

### BRIEF DESCRIPTION OF FIGURES

Arrangements of the present invention will be understood and appreciated more fully from the following detailed description, made by way of example only and taken in conjunction with drawings in which:

FIG. 1A is a block diagram which schematically illustrates, in the upper panel, a system as known in the prior art to process private imaging data based on an inference model and a conventional encryption scheme, and in the lower panel, a system as known in the prior art to process private imaging data based on an inference model and a homomorphic encryption scheme;

FIG. 1B is a block diagram of the system of the lower panel of FIG. 1A, wherein the inference model is an autoencoder;

FIG. 2 is a schematic diagram of an apparatus according to an embodiment;

FIG. 3A is a block diagram which schematically illustrates a system for performing inference on imaging data according to an embodiment;

FIG. 3B is an example of a segmentation result;

FIG. 4 is a block diagram which schematically illustrates a system for performing inference on imaging data based on the UNET model according to an embodiment; and

FIG. 5 is a flow diagram illustrating a method for performing inference on imaging data.

### DESCRIPTION

Certain embodiments provide a medical information processing apparatus comprising a processing circuitry configured to: receive imaging data; apply a first part of an inference model to the imaging data to obtain intermediate analysis results; encrypt the intermediate analysis results based on a homomorphic encryption scheme; and send the encrypted intermediate analysis results to a remote server for processing with a second part of an inference model to result in encrypted analysis results.

Certain embodiments provide a medical information processing method, the method comprising: receiving imaging data; applying a first part of an inference model to the imaging data to obtain intermediate analysis results; encrypting the intermediate analysis results based on a homomorphic encryption scheme; and sending the encrypted intermediate analysis results to a remote server for processing with a second part of an inference model to result in encrypted analysis results.

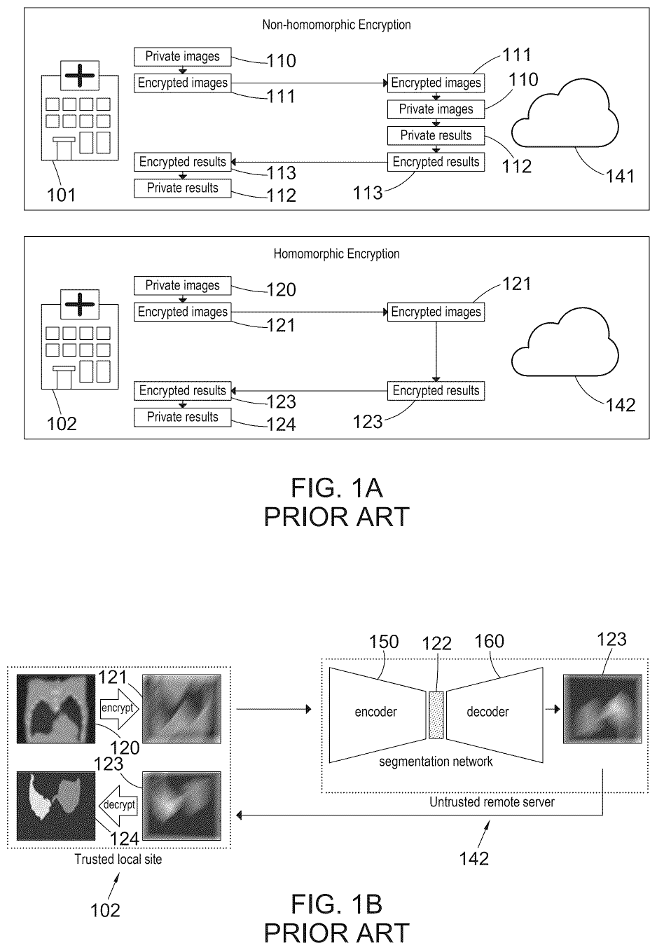

FIG. 1A illustrates two block diagrams which schematically show systems as known in the prior art for the secure transfer and analysis of private imaging data between a trusted local site and an untrusted remote server based on encryption schemes. The private imaging data is analysed using an inference model. The upper panel of FIG. 1A shows a system based on the application of non-homomorphic encryption scheme, and the lower panel of FIG. 1A shows a system based on the application of a homomorphic encryption scheme.

As used herein, imaging data can be private imaging data relating to one or more persons that is not intended to be made available to the general public. In some embodiments, the private imaging data is medical imaging data relating to one or more patients. The medical imaging data can be obtained by any suitable method for imaging a patient as part of a clinical diagnosis procedure by performing methods such as MRI, ultrasound, computed tomography (CT scan), X-ray, or whole slide imaging. The imaging data can be 2D, 3D or a video sequence. The imaging data can refer to raw imaging data obtained directly from an imaging device or raw imaging data that has undergone pre-processing.

Encryption schemes refer to methods which encode an original data representation, known as plaintext, into an alternative form, known as ciphertext. The ciphertext is effectively a scrambled version of the plaintext. An encryption key, which is generated by an algorithm, is used to encode plaintext into ciphertext. A decryption key, which is also generated by an algorithm, is used to decode the ciphertext. Ideally, the private key is only made available to authorized parties. Symmetric encryption refers to encryption schemes where the same key is used for encryption and decryption. Public-key encryption refers to encryption schemes where the encryption key is different to the decryption key. The encryption key is made public while the decryption key is secret.

Typically, ciphertext generated by conventional encryption schemes does not preserve the same structure as the plaintext. As a consequence, a mathematical function performed on ciphertext, and then decoded back to plaintext, will not give a result which is equivalent to the performance of the same mathematical function on plaintext. Homomorphic encryption schemes, by comparison, encode plaintext such that the resulting ciphertext has corresponding structure to the plaintext. As a consequence, a limited set of mathematical functions can be used to transform homomorphically encoded ciphertext which corresponds to original plaintext, and when the transformed ciphertext is decrypted back to plaintext, the result is equivalent to if the same set of mathematical functions were performed on the original plaintext. The limited set of mathematical functions that can be performed on homomorphically encrypted data comprises polynomial functions which are built up of additions and/or multiplications only.

As used herein, an inference model refers to any model or algorithm for obtaining meaning from an image based on a set of input values. With reference to medical image data in particular, an inference algorithm refers to any model for identifying, classifying, segmenting and/or quantifying patterns or pixels in an image for use in clinical analysis and/or diagnosis. For example, the inference algorithm can be an image segmentation algorithm configured to assign a label to each pixel in the image, with each label corresponding to a respective type of tissue. In another example, the inference model can be a classification model configured to determine if an image relates to a clinical category, for example, if the image corresponds to the presence or absence of cancer in a sample.

With reference to the upper panel of FIG. 1A, which illustrates the application of a non-homomorphic encryption scheme, a first trusted local site 101 is configured to receive first private image data 110. The first trusted local site 101 is configured to encrypt the first private image data 110 according to a public-key, non-homomorphic encryption scheme to obtain first encrypted data 111. The first trusted local site 101 is further configured to send the first encrypted data 111 to a first untrusted remote server 141. Separately, the first trusted local site 101 is configured to send a decryption key to the untrusted remote server 141. The first untrusted remote server 141 is configured to decrypt the first encrypted data 111 to obtain the first private image data 110. The first untrusted remote server 141 is further configured to apply an inference model to the first private image data 110 to generate first private analysis results 112. The inference model can be an image classification or image segmentation model. The first untrusted remote server 141 is further configured to encrypt the first private analysis results 112 according to a public-key, non-homomorphic encryption scheme to obtain first encrypted analysis results 113. The first untrusted remote server 141 is further configured to send the first encrypted analysis results 113 to the first trusted local site 101. If a different encryption scheme is used at the remote server 141 compared to at the local site 101, separately, the remote server 141 is configured to send a decryption key to the local site 101. The first trusted local site 101 is configured to decrypt the first encrypted private analysis results 113 to obtain first private analysis results 112.

Turning to the lower panel of FIG. 1A, which illustrates the application of a homomorphic encryption scheme, a second trusted local site 102 is configured to receive second private image data 120. The second trusted local site 102 is configured to encrypt the second private image data 120 according to a public-key homomorphic encryption scheme to obtain second encrypted data 121. The second trusted local site 102 is further configured to send the second encrypted data 121 to a second untrusted remote server 142. The second untrusted remote server 142 is configured to apply an inference model to the second encrypted data 121 to generate second encrypted analysis results 123. The inference model can be an image classification or image segmentation model which is adapted for implementation on homomorphically encrypted data. For example, any non-linear operations in the inference model can be replaced by polynomial approximations comprising additions and multiplications only. The second untrusted remote server 142 is further configured to send the second encrypted analysis results 123 to the second trusted local site 102. The second trusted local site 102 is configured to decrypt the second encrypted private analysis results 123 to obtain second private analysis results 124.

In comparison to the scheme shown in the upper panel of FIG. 1A, it can be seen from the lower panel of FIG. 1A that private analysis results can be generated in fewer steps when private image data is encrypted based on a homomorphic encryption scheme, compared to when a non-homomorphic encryption scheme is used. In addition, there is no requirement to exchange encryption keys between the trusted local site and the untrusted remote site when a homomorphic encryption scheme is used.

The inference model can be represented as a function which receives an image as input and outputs a prediction for the image. The architecture of the inference model can be modular in structure. For example, the inference model can also be represented as two functions, i.e. as two parts. The inference model can be partitioned into a first part and second part such that a first part of the inference model receives input data and performs a first processing of the input data to generate intermediate analysis results, and the second part of the inference model receives the intermediate analysis results and performs a second processing on the intermediate analysis results to output a prediction for the image, which are final analysis results. It will be understood that whilst each of the first part and second part of the inference model can be represented as respective functions, each of the respective functions can comprise more than one sub-function. For example, an inference model such as a neural network comprising many layers or functions can be partitioned into a first part and a second part, each of the first part and second part respectively comprising a plurality of layers, or functions.

FIG. 1B illustrates a block diagram of a system as known in the prior art for the secure transfer and analysis of private imaging data between a trusted local site and an untrusted remote server based on an homomorphic encryption scheme and an inference model divided into two parts. The components of the system illustrated in FIG. 1B are equivalent to the components of the system illustrated in the lower panel of FIG. 1A, except for that the untrusted remote server of FIG. 1B is specifically configured to apply an inference model known as an autoencoder. An autoencoder comprises two functions, an encoder 150 and a decoder 160. The encoder 150 can be considered to be the first part of an inference model, and the decoder 160 can be considered to be the second part of an inference model. The encoder 150 is configured to transform second encrypted data 121 into intermediate analysis results 122, which is a latent representation of the second encrypted data. The latent representation is typically of lower dimensionality than the second encrypted data 121. The decoder 160 is configured to transform the intermediate analysis results 122 to a reconstruction of the second encrypted data 121. The encoder 150 and decoder 160 can each be neural networks. The decoder 160 can be trained to perform image segmentation, such that the output of the decoder 160 comprises homomorphically encrypted image segmentation information relating to the second private image data 120. The output of the decoder is the second encrypted analysis results 123.

There are challenges associated with performing the entirety of an inference model homomorphically encrypted data, as shown in FIG. 1B, since it can be prohibitively expensive. The encryption process itself, as well as the deployment of inference models on homomorphically encrypted data, can be slow and computationally expensive. In addition, only polynomial operations such as operations comprising addition and/or multiplication can be performed on homomorphically encrypted data. This means that operations such as activation functions, which are a common feature of artificial intelligence methods, must be approximated by polynomial functions which can result in a reduction in accuracy of the inference model.

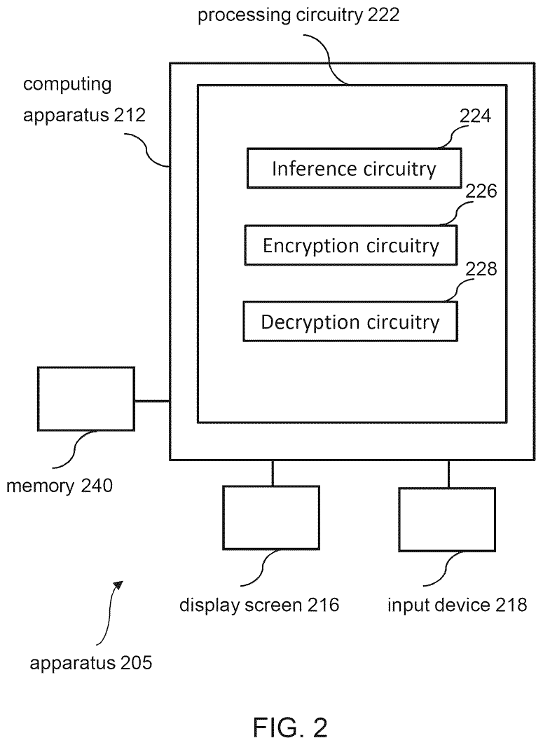

An apparatus 205 according to an embodiment will now be described with reference to FIG. 2. The apparatus 205 may also be referred to as a medical information processing apparatus. The apparatus 205 is configured to process imaging data, which can be private medical imaging data. The apparatus 205 is configured to apply the first part of an inference model to unencrypted imaging data to obtain intermediate analysis results. The apparatus is further configured to encrypt the intermediate results according to a homomorphic encryption scheme and send the encrypted intermediate analysis results to a remote server, where the second part of an inference model is applied to the encrypted intermediate results. In other embodiments, the apparatus 205 may be configured to process any appropriate data.

The apparatus 205 comprises a computing apparatus 212, which can be a computer or server. The computing apparatus 212 can be connected to a display screen 216 or other display device. The computing apparatus is further connected to an input device or devices 218, such as a computer keyboard and mouse. The computing apparatus 212 receives data from memory 240, which may also be referred to as a data store or storage. The memory 240 stores imaging data and a first part of an inference model. The memory 240 further stores software for performing public-key homomorphic encryption.

In alternative embodiments, computing apparatus 212 receives imaging data from one or more further data stores (not shown) instead of or in addition to memory 240. For example, the computing apparatus 212 may receive imaging data from one or more remote data stores (not shown), which may comprise cloud-based storage.

Computing apparatus 212 comprises a processing circuitry 222 for processing data. The processing circuitry 222 comprises a central processing unit (CPU) and Graphical Processing Unit (GPU). The processing circuitry 222 provides a processing resource for automatically or semi-automatically processing imaging data

The processing circuitry 222 comprises an inference circuitry 224 for applying a first part of an inference model to imaging data, an encryption circuitry 224 for encrypting the partially analysed imaging data based on a homomorphic encryption scheme and for sending the encrypted partially analysed imaging data for further analysis at a remote server, and a decryption circuitry 228 for receiving encrypted analysis results from the remote server and decrypting encrypted analysis results.

In the present embodiment, the circuitries 224, 226, and 228 are each implemented in the CPU and/or GPU by means of a computer program having computer-readable instructions that are executable to perform the method of the embodiment. In other embodiments, the circuitries may be implemented as one or more ASICs (application specific integrated circuits) or FPGAs (field programmable gate arrays).

The computing apparatus 212 also includes a hard drive and other components of a computer including RAM, ROM, a data bus, an operating system including various device drivers, and hardware devices including a graphics card. Such components are not shown in FIG. 2 for clarity.

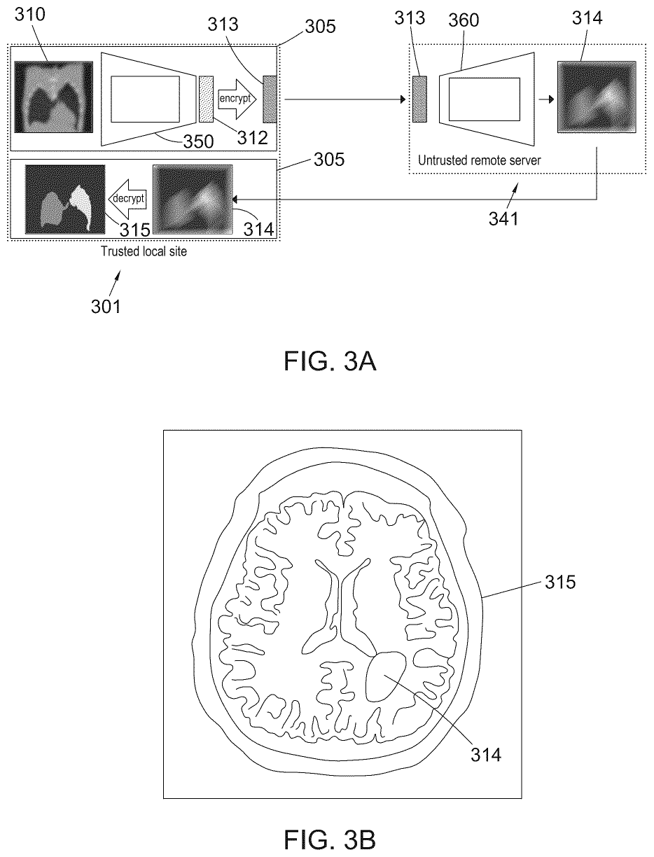

The apparatus 205 of FIG. 2 can be implemented as part of a system 300 for the secure transfer and analysis of private imaging data based on a homomorphic encryption scheme as illustrated in FIG. 3A. The system 300 comprises a trusted local site 301 and untrusted remote server 341. The trusted local site 301 comprises apparatus 305, which corresponds to apparatus 205. The apparatus 305 stores a first part of an inference model 350. In some embodiments, the apparatus 305 is implemented by more than one computing device and each of the more than one computing device are connected to each other over a secure network. In some embodiments, the trusted local site 301 is a computer network of a hospital, medical institution, or research institution.

The untrusted remote server 341 stores a second part of the inference model 360. In some embodiments, the untrusted remote server 341 is implemented by more than one computing device. In some embodiments, the untrusted remote server 341 is based on the cloud. The trusted local site 301 does not have privileges to access the untrusted remote server 341, and the untrusted remote server 341 does not have privileges to access the trusted local site 301. In some embodiments, data transfer between the trusted local site 301 and the untrusted remote server 340 can be implemented using a file sharing service such as Dropbox, a secure file sharing service such as Box or FFP, or a network protocol such as SSH.

The first and second parts of the inference model 350, 360 combined constitute a function which takes imaging data as input and provides a prediction as output. The first part of an inference model 350 is configured to process image data before it is encrypted. The first part of the inference model 350 can therefore comprises non-polynomial functions such as activation functions. The second part of the inference model 360 is configured to process homomorphically encrypted data. The second part of the inference model 360 therefore only comprises additions and multiplications. In some embodiments, the second part of the inference model 360 comprises a polynomial approximation of a function that comprises non-polynomial operations, wherein the polynomial approximation comprises additions and/or multiplications only.

In addition to this, or alternatively, in some embodiments, the inference model is partitioned into first and second parts 350, 360 such that a proprietary model is applied to the encrypted data only. For example, the first part of the inference model 350 hosted at the trusted local site 301 can be a publically available model, which can be an open source model. In some embodiments, the first part of the inference model 350 can be a generic feature extractor such as ResNet.

The second part of the inference model 360 can be a proprietary model which is hosted at the remote server 341 and is not publically available. The second part of the inference model can be custom made for providing predictions on certain types of medical imaging data. For example, the second part of the inference model 360 can be customized for a certain type inference task on a specific type of imaging data. For example, the second part of the inference model 360 can be customized for performing segmentation tasks on MRI brain data.

In addition to this, or alternatively, in some embodiments, the inference model is partitioned into first and second parts 350, 360 such that the computationally intensive tasks are performed on the unencrypted data. For example, the first part of inference model 350 hosted at the trusted local site 301 comprises computationally intensive tasks which can more quickly be performed on raw imaging data. The second part of the inference model 360 hosted at the remote server 341 comprises less computationally intensive operations which reduces the computational burden of analysing homomorphically encrypted data. The first part of inference model can comprise a first plurality of neural network layers and the second part of the inference model can comprise a second plurality of neural network layers. The first plurality of neural network layers can be greater than the second plurality of neural network layers. In addition, or alternatively, the first part of the inference model can comprise a first number of operations and second part of the inference model can comprise a second number of operations, wherein the first number of operations is greater than the second number of operations.

Considering FIG. 3A in further detail, the apparatus 305 is configured to receive imaging data 310, which can be private medical imaging data. The imaging data 310 may have been generated an imaging device connected to the apparatus 305.

Alternatively, the imaging data 310 may have been generated by another imaging device which is part of the trusted local site 301. Alternatively, the imaging data 310 may have been generated at a location remote to the trusted local site 301 and securely sent to a server of the trusted local site 301.

The apparatus 305 is configured to apply the first part of an inference model 350 to the imaging data 310 to obtain an intermediate analysis results 312. The apparatus is further configured to encrypt the intermediate analysis results 312 based on a homomorphic encryption scheme to obtain encrypted intermediate analysis results 313. The homomorphic encryption scheme can be a levelled homomorphic encryption scheme or a fully homomorphic encryption scheme. The intermediate analysis results 312 can be encrypted based on a public key generated using a public-key homomorphic encryption scheme. A private key, which is paired to the public key, is also generated based on the same public-key homomorphic encryption scheme which is used to generate the public key.

The homomorphic encryption scheme can be based on the linear algebra problem Learning with Errors (LWE) and the extension Ring Learning with Errors (RLWE). This scheme is based on solving the linear system of equations As+e=b, where A and s are the secret keys, s is a small error term and b is the public key. In LWE, the entries to the matrices are integers modulo some prime, p. In the RLWE the entries are polynomials with degree (p−1). The larger the prime, the more complex the scheme, making it more secure but also more complex and computationally expensive. Both private and public keys can be obtained by sampling from valid vectors in the chosen space based on a uniform sampling or based on a chosen distribution.

The apparatus 305 is configured to store the private key. The apparatus 305 is further configured to send the encrypted intermediate analysis results 313 to the untrusted remote server 341.

In some embodiments, the public-key homomorphic encryption scheme is a Cheon, Kim, Kim and Song (CKKS) scheme, which allows additions and multiplications to be performed on the encrypted data The CKKS encryption scheme may be implemented using a library such as HEAAN (Cryptolab), SEAL (Microsoft), or HeLib (IBM).

In other embodiments, the public-key homomorphic encryption scheme is a Brakerski-Fan-Vercauteren (BFV) scheme, or Brakerski-Gentry-Vaikuntanathan scheme, which allows modular arithmetic to be performed on encrypted integers. In these embodiments, it would be required to convert the values in the input data to integer values. The BFV scheme may be implemented using a library such as SEAL (Microsoft), PALISADE (New Jersey Institute of Technology), or Lattigo (Laboratory for Data Security). The BGV scheme may be implemented using a library such as PALISADE (New Jersey Institute of Technology), HELib (IBM), or Lol (Crockett and Peikert).

The untrusted remote server 341 is configured to receive the encrypted intermediate analysis results 313 and apply the second part of the inference model 360 to the encrypted intermediate analysis results 313 to obtain encrypted analysis results 314. The untrusted remote server 341 is configured to send the encrypted analysis results 314 to the apparatus 305. The apparatus 305 is configured to decrypt the encrypted analysis results 314 using the private key to obtain private analysis results 315.

In some embodiments, different servers at the medical site can perform different processing steps on the imaging data. For example, a first apparatus 305 can be an imaging server for applying the first part of an inference model on the imaging data. The same server, or a different server 305 to the one which performed the image analysis, can be used to generate the public-private key pair and encrypt the intermediate analysis results. A different server 305 can be used to receive the encrypted analysis results 314, decrypt them, and store them. In such embodiments, a key exchange is performed between the server 305 which generated the public-private key pair, and the server 305 which decrypts the encrypted analysis results 314. The server 305 which generate the public-private key pair is configured to send the private key to the server 305 which decrypts the encrypted analysis results 314.

In embodiments where the imaging data 310 comprises a plurality of separate images 310, the apparatus 305 is configured to apply a first part of an inference model 350 to each of the plurality of images to obtain a plurality of intermediate analysis results 312. The apparatus 305 is further configured to separately encrypt each of the plurality of intermediate analysis results 312 and send each of the plurality of encrypted intermediate analysis results 313 to the untrusted remote server 341. The untrusted remote server 341 is configured to apply the second part of the inference model 360 to each of the plurality of encrypted intermediate analysis results 313 to obtain a plurality of encrypted analysis results 314. The apparatus 305 is configured to decrypt each of the plurality of encrypted analysis results 314 to obtain a plurality of analysis results 315.

It will be understood that whilst, in the present embodiment, the second part of inference model 360 is applied to homomorphically encrypted data, the second part of the inference model 360 can also be usefully applied to unencrypted data. For example, the second part of the inference model 360 could be applied to the intermediate analysis results 312, which are unencrypted, to provide results which are equivalent to the analysis results 315.

The first and second parts of the inference model can be respectively trained using training data comprising medical images and corresponding ground truth data. During training, the parameters of the respective first and second parts of the inference model are modified to minimize a loss function. When training for segmentation tasks, the ground truth data comprises contours or regions of the corresponding medical image which respectively relate to a specific tissue type or pathology. When training for classification tasks, the ground truth data comprises a classification of the corresponding medical image. The ground truth data can be manually defined by clinical experts or generated by software. The training of the respective first and second parts of the inference model can be performed separately. The first and second parts of the inference model can be trained on the same, or different, set of training data. The first part of the inference model can be trained at the trusted local site or elsewhere. The second part of the inference model can be trained at the untrusted remote server. In some embodiments, private medical data is used to train the first part of the inference model at the trusted local site.

In some embodiments, the inference model is an autoencoder and the first part of the inference model 350 is an encoder and the second part of the inference model 360 is a decoder. In these embodiments, the intermediate analysis results 312 is a latent space representation of the imaging data 310. The encrypted analysis results 314 is an encryption of a segmented version of the imaging data 310. The analysis results 315 is a segmented version of the imaging data 310. analysis results 315 comprising a segmentation of image data 310, which is MRI imaging data of a brain. The analysis results 315 comprise an image region 320 corresponding to predicted brain tumour. It will be appreciated that the inference architecture disclosed herein can be modified to perform inference on any type of medical imaging data and a plurality of different properties may be inferred from the data. For example, different types of abnormal tissue may be inferred.

In other embodiments, the inference model is a vision transformer (not shown). A vision transformer, such as the vision transformer described in Dosovitskiy, Alexey, et al. “An image is worth 16×16 words: Transformers for image recognition at scale.” arXiv preprint arXiv: 2010.11929 (2020), is configured to receive an image as input and generate a classification of an image as output.

The vision transformer comprises a first module configured to receive an image as input and generate patches of the image. The vision transformer further comprises a second module configured to generate patch and position embeddings corresponding to the patches of the input image. The vision transformer comprises a transformer encoder configured to receive the patch and position embeddings of the input image and output a latent space representation of the patch and position embeddings. The vision transformer further comprises a multilayer perceptron (MLP) head configured to receive the latent space representation and output a classification of the image data.

According to an embodiment, a vision transformer can be partitioned into a first part 350 and second part 360 as follows. The first part of the inference model 350 comprises the first module configured to receive imaging data 310 and generate image patches, the second module configured to receive image patches and generate the patch and position embeddings, and a transformer encoder configured to receive the patch and position embeddings and generate a latent space representation of the patch and position embeddings. The second part of the inference model 350 comprises the MLP head configured to receive the latent space representation of the patch and position embeddings and output a classification. In these embodiments, the intermediate analysis results 312 is a latent space representation of the patch and position embeddings of the patches of the imaging data 310. The encrypted analysis results 314 comprises an encryption of a classification of the imaging data 310. The analysis results 315 comprise a classification of the imaging data 310.

In some embodiments, the first part of the inference model 350 can comprise a generic transformer and the second part of the inference model 360 can comprise a custom MLP head specific for an encrypted latent space of medical imaging data. Since transformers are typically trained on large public datasets, a generic transformer can be effectively applied to the imaging data. By comparison, the MLP head can be adapted by a third party for a specific classification problem. The MLP can be kept secret by hosting the MLP head at the untrusted remote server 341.

The transformer encoder is held on the trusted local site and operates on the raw, unencrypted data while the more custom, project specific MLP head is held on the remote server. The transformer encoder is made up of L identical pairs of layers. In each pair there is a self-attention layer which is extracts features from each patch, considering the neighbouring patches, as well as a feed forward layer used to non-linearly transform the output of the attention layers. Like the encoder-decoder architectures described above, a deep encoder in a transformer may help it solve more complex problems. However, it very quickly become very computationally expensive. The MLP head used for classification is usually a simpler network, consisting of a smaller number of layers. The types of layers included in the MLP are fully connected layers and activation functions.

In some embodiments, the vision transformer can be applied to segmentation, rather than classification tasks. In these embodiments, the MLP head is replaced by a decoder or upsampling network, and the analysis results 315 comprise a segmentation of the imaging data 310.

In some embodiments, the transformer is trained on a large, more general dataset and then fine-tuned for the specific task on higher quality data of greater relevance to the inference task to be performed. The transformer may be fine-tuned at the local site using private data comprising relevant medical imaging data and corresponding ground truth data. However, for security, it would be preferable that any model fine-tuned on private hospital data would not leave the local site. The MLP head (or decoder and upsampling network) can be first trained on a large more general dataset and then fine-tuned for the specific task on higher quality data. Since the MLP head for classification (or decoder or upsampling network for segmentation) is on a remote server, it cannot be fine-tuned on private data from the secure site. Instead, the MLP head (or decoder or upsampling network) can be fine-tuned using the model owner's data, which comprises medical imaging data and corresponding ground truth data.

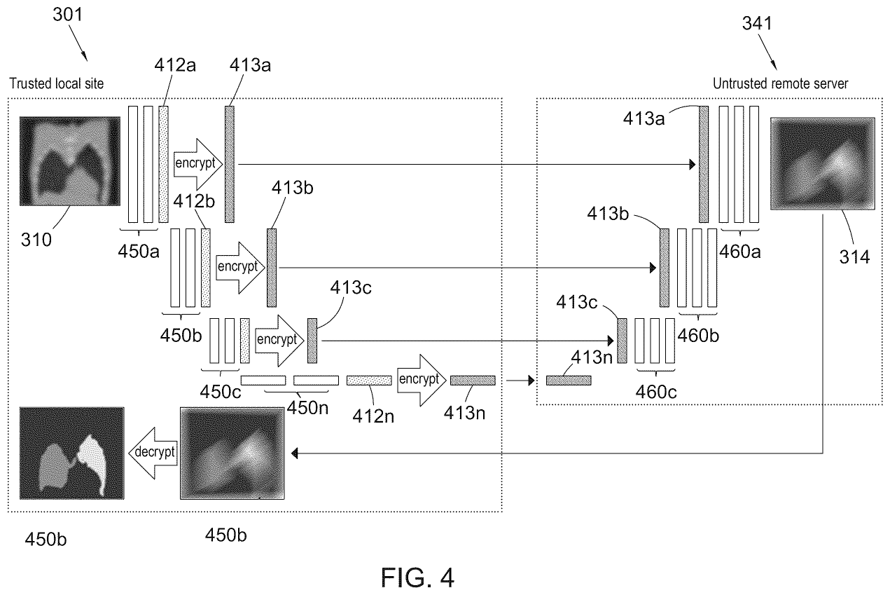

Turing to FIG. 4, in further embodiments, the inference model is an image segmentation model such as UNET, which is described in Ronneberger, Olaf, Philipp Fischer, and Thomas Brox. “U-net: Convolutional networks for biomedical image segmentation.” Medical Image Computing and Computer-Assisted Intervention-MICCAI 2015: 18th International Conference, Munich, Germany, October 5-9, 2015, Proceedings, Part III 18. Springer International Publishing, 2015. In these embodiments, the architecture of the inference model comprises a contracting path comprising encoder layers, and an expansive path comprising decoder layers. In these embodiments, the first part of the inference model 350 comprises encoder layers 450a-n. The second part of the inference model 360 comprises decoder layers 460a-n. The first encoder layer 450a is configured to receive the imaging data 310 and output a latent representation of the imaging data 310, which will be referred to as intermediate analysis results 512a. Each of the second to nth encoder layers 450b-n are configured to receive an input based on the output of a preceding encoder layer, and output a latent representation of the input, which will be referred to as intermediate analysis results 412b-n. The apparatus 305 is configured to encrypt each of the intermediate analysis results 412a-b to obtain a plurality of encrypted intermediate analysis results 413a-n.

The remote server 341 is configured to receive each of the plurality encrypted intermediate analysis results 413a-n. Each of the decoder layers 460a-n are configured to receive a corresponding encrypted intermediate analysis result 413a-n of the plurality encrypted intermediate analysis results 413a-n, which is provided by an architecture known as a skip connection. Each of the decoder layers 460a-n are further configured to receive an output from succeeding decoder layer, except for the final decoder layer 460n. Each of the decoder layers 460a-n are configured to generate an output (not shown) based on their inputs. The final decoder layer 460n is not configured to apply any transformation to its input, however the decoder layers 460a-460n-1 are configured to transform their respective inputs. For example, decoder layer 460n receives 413n only and generates an output 413n. Decoder layer 460b receives 413b and the output of decoder layer 460c as input, and generates an output which is a transformation of its inputs. Decoder layer 460a receives 413b and the output of decoder layer 460b as input, and generates an output which is a transformation of its inputs. The output of the first decoder layer 460a comprises the encrypted analysis results 314 which is an encryption of segmented imaging data 310. The analysis results 315 are segmented imaging data 310.

In some embodiments, each of the encoder layers 450 a-n comprise two consecutive 3×3 convolutions, followed by a rectified linear unit (ReLU) function. Each of the second to nth encoder layers 450b-n are configured to receive the output of a preceding encoder layer which has had a 2×2 max pooling operation with stride 2 applied to it, with the number of feature channels doubled. Each of the skip layers comprise the a copy and crop function. Each of the first to (n−1)th decoder layers 460a-n-1 are configured to receive an output from succeeding decoder layer which has had a 2×2 up-convolution applied to it which halves the number of feature channels. Each of the first to (n−1)th decoder layers comprise two consecutive 3×3 convolutions, followed by a rectified linear unit (ReLU) function. The first decoder layer 460a further comprises a 1×1 convolution layer.

In further embodiments, the encrypted intermediate analysis results 313 can be hosted on a data custodian server. The encrypted intermediate analysis results 313 can be made available for research or commercial purposes. The encrypted intermediate analysis results 313 can be offered to customers, or users, as service. In one embodiment, customer devices can make queries of this data using an API. Since the encrypted intermediate analysis results 313 are homomorphically encrypted, patient privacy is protected, whilst allowing analysis to be performed on the encrypted intermediate analysis results 313.

In further embodiments, in order to increase the efficiency of computation performed by the apparatus 305 in performing homomorphic encryption, the apparatus 305 utilises a cryptographic accelerator which is a co-processor specifically designed to perform computationally intensive cryptographic computation. In further embodiments, to increase the efficiency of computation performed by the untrusted server on the encrypted data, the remote server utilizes an accelerator which is a co-processor specifically designed to perform computationally intensive tasks on homomorphically encrypted data. In addition to this, or alternatively, a Graphical Processing Unit (GPU) implementation can be used at the trusted local site 301 and/or the remote server 341 to decrease the run-time.

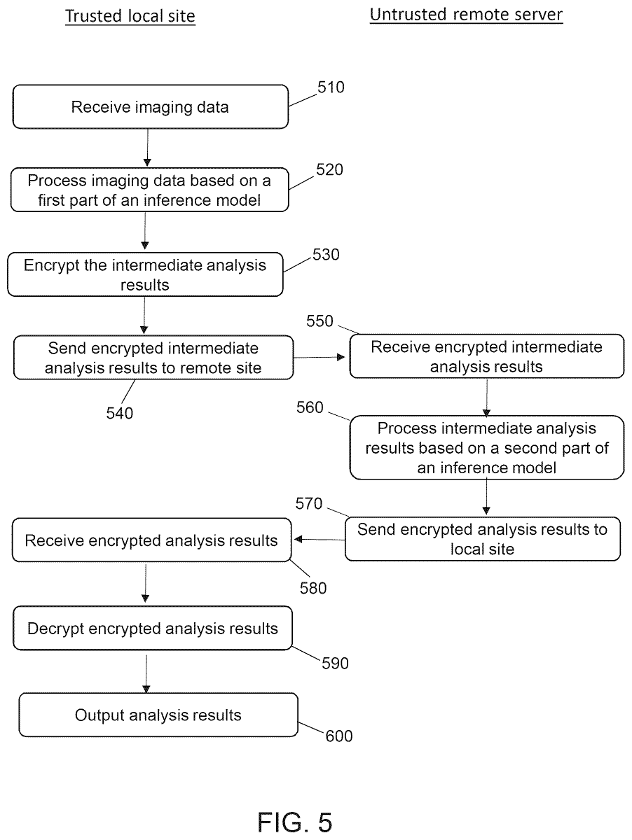

Turning to FIG. 5, a method for the secure transfer and analysis of imaging data will now be described. At 510, the apparatus 305 receives imaging data 310. At 520, the apparatus applies the first part of an inference model 350 to the imaging data 310 to obtain an intermediate analysis results 312. At 530, the apparatus generates a public-private key pair based on a homomorphic encryption scheme. The apparatus 305 encrypts the intermediate analysis results 312 using the public key to obtain encrypted intermediate analysis results 313. At 540, the apparatus 305 sends the encrypted intermediate analysis results 313 to the untrusted remote server 341.

At 550, the untrusted remote server 341 receives the encrypted intermediate analysis results 313. At 560, the untrusted remote server 341 applies the second part of the inference model 360 to the encrypted intermediate analysis results 313 to obtain encrypted analysis results 314. At 570, the untrusted remote server 341 sends the encrypted analysis results 314 to the apparatus 305.

At 580, the apparatus 305 receives the encrypted analysis results 314. At 590, the apparatus 305 decrypts the encrypted analysis results 314 using the private key to obtain private analysis results 315. Optionally, if the apparatus 305 which performs the decryption is different to the apparatus 305 which performs the encryption, the private key is first sent from the apparatus 305 which generated the public-key pair to the apparatus 305 which performs the decryption.

At 600, the apparatus 305 outputs the private analysis results 315. The private analysis results 315 can optionally be displayed on a monitor for inspection by a clinician or researcher.

Advantageously, the systems and methods described here provide a way for homomorphically encrypted patient data to be analysed by a proprietary model which reduces both the run-time and computational resource usage when compared to methods which apply inference methods to homomorphically encrypted data only. At the same time, the systems and methods described herein allow for the IP of proprietary models and patient privacy to be protected. This is achieved by dividing an inference model between the trusted local site and an untrusted remote server so that only necessary computations are performed encrypted data. For example, a generic model, such as a generic encoder, is applied on the raw patient data at the trusted local site. Operations performed on unencrypted data will be faster and less resource intensive than on encrypted data. Additionally, non-linearity can be included on operations performed on the unencrypted data. This avoids any loss of accuracy that may occur through approximating non-linear functions with additions and subtractions, which would be required if the data were homomorphically encrypted. The output of the generic model, such as a generic encoder, is encrypted and sent to the remote server where a proprietary model, such as a custom decoder is stored. In doing so, no sensitive patient information leaves the trusted site. The analysis which is performed locally on the unencrypted data does not violate the IP rights of the owner of the proprietary model. The solution provided herein allows for a medical provider to not be limited to only a subset of models which are available for local use. The solution provided herein allows for the owner of a proprietary model so productise their model, for example by implementing a pay per use approach.

Certain embodiments provide a medical imaging method where the first section of an inference model is used to perform computations on unencrypted data on a secure, trusted server and the output is encrypted by a homomorphic encryption scheme; and wherein the second part of the model, stored on a remote server, is executed on the homomorphic-encrypted output.

The model can be used for medical image segmentation. The model can have an encoder-decoder architecture with the encoder held locally and the decoder on the remote server. The images can be encoded using a generic, off-the-shelf feature detector such as a ResNet. The data can be encrypted using a levelled homomorphic encryption scheme. The data can be encrypted using a fully homomorphic encryption scheme. The model can be divided to minimise the calculations performed on the remote server. The model can be divided such that only the final layers of the network are put on the remote server. The model can be divided such that the more complex and costly calculations are performed on the local trusted server. The first part of the model stored on the local server can contain non-linear functions. The run-time of the method can be reduced by using a GPU implementation.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

According to various embodiments there is provided a medical information processing apparatus comprising a processing circuitry configured to: receive imaging data; apply a first part of an inference model to the imaging data to obtain intermediate analysis results; encrypt the intermediate analysis results based on a homomorphic encryption scheme; and send the encrypted intermediate analysis results to a remote server for processing with a second part of an inference model to result in encrypted analysis results.

Optionally, the processing circuitry is further configured to: receive the encrypted analysis results from the remote server; and decrypt the encrypted analysis results to obtain analysis results.

Optionally, the inference model comprises a first part and a second part, wherein the inference model is configured to receive image data as input and provide an analysis result based on the imaging data as output, wherein the analysis result comprises a classification of the image data and/or segmentation of the image data.

Optionally, the first part of the inference model is a publically available model.

Optionally, the first part of the inference model comprises an encoder.

Optionally, the first part of the inference model comprises non-linear operations.

Optionally, the second part of the inference model is a proprietary model.

Optionally, the second part of the inference model comprises a decoder.

Optionally, the second part of the inference model does not comprise non-linear operations.

Optionally, the second part of the inference model comprises addition and/or multiplication operations only.

Optionally, each of the first part of the inference model and the second part of the inference model comprises a plurality of neural network layers, wherein the second part of the inference model comprises fewer neural network layers than the first part of the inference model.

Optionally, the first part of the inference model comprises a greater number of operations than a number of operations in the second part of the inference model.

Optionally, the processing circuitry is configured to encrypt the intermediate analysis results by: generating a public-private key pair according to a homomorphic encryption scheme; and encrypting the intermediate analysis results based on the public key.

Optionally, the processing circuitry is configured to decrypt the encrypted analysis results by decrypting the encrypted analysis results based on the private key.

Optionally, the medical information processing apparatus comprises one or more first servers and one or more second servers, wherein the step of generating a public-private key pair is performed at a sever of the one or more first servers, and the step of decrypting the analysis results is performed at a server of the one or more second servers, and wherein the processing circuitry is further configured to send the private key from the server of the one or more first servers to the server of the one or more second servers.

Optionally, the imaging data is private medical imaging data.

Optionally, the medical information processing apparatus is connected to a secure network of a hospital or medical research institution.

Optionally, the homomorphic encryption scheme is a levelled homomorphic encryption scheme or a fully homomorphic encryption scheme.

Optionally, the medical information processing apparatus utilizes a GPU implementation.

Optionally the medical information processing apparatus utilizes a cryptographic accelerator.

According to various embodiments there is provided a medical information processing method, the method comprising: receiving imaging data; applying a first part of an inference model to the imaging data to obtain intermediate analysis results; encrypting the intermediate analysis results based on a homomorphic encryption scheme; sending the encrypted intermediate analysis results to a remote server for processing with a second part of an inference model to result in encrypted analysis results.

Optionally the method further comprises: receiving the encrypted analysis results from the remote server; and decrypting the encrypted analysis results to obtain analysis results.

1. A medical information processing apparatus comprising a processing circuitry configured to:
receive imaging data;
apply a first part of an inference model to the imaging data to obtain intermediate analysis results;
encrypt the intermediate analysis results based on a homomorphic encryption scheme; and
send the encrypted intermediate analysis results to a remote server for processing with a second part of an inference model to result in encrypted analysis results.

2. The medical information processing apparatus of claim 1, wherein the processing circuitry is further configured to:
receive the encrypted analysis results from the remote server; and
decrypt the encrypted analysis results to obtain analysis results.

3. The medical information processing apparatus of claim 1, wherein the inference model comprises a first part and a second part, wherein the inference model is configured to receive image data as input and provide an analysis result based on the imaging data as output, wherein the analysis result comprises a classification of the image data and/or segmentation of the image data.

4. The medical information processing apparatus of claim 1, wherein the first part of the inference model is a publically available model.

5. The medical information processing apparatus of claim 1, wherein the first part of the inference model comprises an encoder.

6. The medical information processing apparatus of claim 1, wherein the first part of the inference model comprises non-linear operations.

7. The medical information processing apparatus of claim 1, wherein the second part of the inference model is a proprietary model.

8. The medical information processing apparatus of claim 1, wherein the second part of the inference model comprises a decoder.

9. The medical information processing apparatus of claim 1, wherein the second part of the inference model does not comprise non-linear operations.

10. The medical information processing apparatus of claim 1, wherein the second part of the inference model comprises addition and/or multiplication operations only.

11. The medical information processing apparatus of claim 1, wherein each of the first part of the inference model and the second part of the inference model comprises a plurality of neural network layers, wherein the second part of the inference model comprises fewer neural network layers than the first part of the inference model.

12. The medical information processing apparatus of claim 1, wherein the first part of the inference model comprises a greater number of operations than a number of operations in the second part of the inference model.

13. The medical information processing apparatus of claim 2, wherein the processing circuitry is configured to encrypt the intermediate analysis results by:
generating a public-private key pair according to a homomorphic encryption scheme; and
encrypting the intermediate analysis results based on the public key.

14. The medical information processing apparatus of claim 13, wherein the processing circuitry is configured to decrypt the encrypted analysis results by decrypting the encrypted analysis results based on the private key.

15. The medical information processing apparatus of claim 14, wherein the medical information processing apparatus comprises one or more first servers and one or more second servers, wherein the step of generating a public-private key pair is performed at a sever of the one or more first servers, and the step of decrypting the analysis results is performed at a server of the one or more second servers, and wherein the processing circuitry is further configured to send the private key from the server of the one or more first servers to the server of the one or more second servers.

16. The medical information processing apparatus of claim 1, wherein the imaging data is private medical imaging data.

17. The medical information processing apparatus of claim 1, wherein the medical information processing apparatus is connected to a secure network of a hospital or medical research institution.

18. The medical information processing apparatus of claim 1, wherein the homomorphic encryption scheme is a levelled homomorphic encryption scheme or a fully homomorphic encryption scheme.

19. The medical information processing apparatus of claim 1, wherein the medical information processing apparatus utilizes a GPU implementation.

20. The medical information processing apparatus of claim 1, wherein the medical information processing apparatus utilizes a cryptographic accelerator.

21. A medical information processing method, the method comprising:
receiving imaging data;
applying a first part of an inference model to the imaging data to obtain intermediate analysis results;
encrypting the intermediate analysis results based on a homomorphic encryption scheme; and
sending the encrypted intermediate analysis results to a remote server for processing with a second part of an inference model to result in encrypted analysis results.

22. The medical information process method of claim 21 further comprising:
receiving the encrypted analysis results from the remote server; and
decrypting the encrypted analysis results to obtain analysis results.
